(19) United States
(12) Patent Application Publication
(10) Pub. No.: US 2020/0243179 A1
(43) Pub. Date: Jul. 30, 2020

(54) DATA PROCESSING DEVICE, DATA PROCESSING METHOD, AND NON-TRANSITORY RECORDING MEDIUM STORING THEREIN DATA PROCESSING PROGRAM

(71) Applicants: OMRON Corporation, Kyoto (JP); OMRON HEALTHCARE Co., Ltd., Kyoto (JP)

(72) Inventors: Hiroshi NAKAJIMA, Kyoto (JP); Hirotaka WADA, Kyoto (JP); Tamio UEDA, Kyoto (JP); Daisuke NOZAKI, Kyoto (JP)

(21) Appl. No.: 16/850,431

(22) Filed: Apr. 16, 2020

Related U.S. Application Data
(63) Continuation of application No. PCT/JP2018/031311, filed on Aug. 24, 2018.

(30) Foreign Application Priority Data
Oct. 26, 2017 (JP) 2017-207134

(51) Int. Cl. G16H 15/00; G16H 50/20; G06K 9/00; A61B 5/021

(52) U.S. Cl. CPC G16H 15/00; G16H 50/20; G06K 2209/05; A61B 5/021; G06K 9/00885

(57) ABSTRACT

Provided is a technology that reduces the amount of biometric data sent. A data processing device comprises a biometric data acquisition unit that acquires biometric data of a subject; a sending condition acquisition unit that acquires a sending condition that comprises a first time period and associated with a health-related characteristic of the subject; a data classification unit that determines whether a measurement time of the biometric data is within the first time period, classifies the biometric data as data for sending when a determination result indicates that the measurement time is within the first time period, and classifies the biometric data as data not for sending when the determination result indicates that the measurement time is within a second time period, which is different from the first time period; and a data output unit that outputs the data for sending.

### CROSS-REFERENCE TO RELATED APPLICATIONS

This is a continuation of International Application 2017-207134 with an international filing date of Oct. 26, 2017, and PCT/JP2018/031311 with an international filing date of Aug. 24, 2018 and filed by applicant, the disclosure of which is hereby incorporated by reference in its entirety.

### TECHNICAL FIELD

The present invention relates to processing technology for biometric data.

### BACKGROUND ART

As described in JP 2017-023546 A for example, the development of wearable blood pressure monitors that can measure blood pressure anywhere has been advancing.

There is hope that biometric data obtained under various circumstances can, for example, be used to help determine abnormalities in the body of a subject. One approach taken is to store the biometric data of a plurality of subjects in a server.

### SUMMARY OF INVENTION

In such a case, the server is always receiving and collecting biometric data from a plurality of subjects. However, the total amount of biometric data of the plurality of subjects is huge. Server storage capacity has limits, and storing all of the blood pressure data measure by blood pressure monitors on a server is not plausible.

The present invention provides a data processing device, a data processing method, and a non-transitory recording medium storing therein a data processing program capable of reducing the amount of biometric data sent.

A data processing device according to a first aspect of this invention includes: a biometric data acquisition unit that acquires biometric data of a subject; a sending condition acquisition unit that acquires a sending condition that includes a first time period and associated with a health-related characteristic of the subject; a data classification unit that determines whether a measurement time of the biometric data is within the first time period, classifies the biometric data as data for sending when a determination result indicates that the measurement time is within the first time period, and classifies the biometric data as data not for sending when the determination result indicates that the measurement time is within a second time period, which is different from the first time period; and a data output unit that outputs the data for sending.

According to the first aspect of this invention, the data processing device can output, to the sending destination device, the biometric data of the subject, from among biometric data pieces, that satisfies the sending condition and analyzed by a sending destination device. Thus, the sending destination device stores the biometric data that satisfies the sending condition and is analyzed but does not store the biometric data that does not satisfy the sending condition and is not analyzed. In this manner, the storage capacity of the sending destination device can be effectively used.

The data processing device according to a second aspect of this invention is the data processing device of the first aspect further including a determination unit that determines the health-related characteristic on the basis of the biometric data; and wherein the sending condition acquisition unit acquires the sending condition associated with the health-related characteristic determined by the determination unit.

According to the second aspect of the invention, the data processing device can determine the characteristic related to the current health of a subject on the basis of the biometric data that changes over time due to the lifestyle habits of the subject. Thus, the sending destination device can store the biometric data that satisfies the sending condition associated with the characteristic relating to the current health of a subject.

The data processing device according to a third aspect of this invention is the data processing device of the first aspect, further comprising an instruction output unit that acquires a measurement schedule of the biometric data and outputs an instruction to stop measurement for the biometric data in the second time period in a case where at least a portion of the second time period is within the measurement schedule.

According to the third aspect, the data processing device can stop measurement for biometric data that does not satisfy the sending condition and is not analyzed by the sending destination device. Thus, the load on measurement processing of the biometric data and the load on classification processing of the biometric data are reduced.

A data processing method according to a fourth aspect of the invention includes: acquiring biometric data of a subject; acquiring a sending condition that includes a first time period and associated with a health-related characteristic of the subject; determining whether a measurement time of the biometric data is within the first time period, classifying the biometric data as data for sending when a determination result indicates that the measurement time is within the first time period, and classifying the biometric data as data not for sending when the determination result indicates that the measurement time is within a second time period different from the first time period; and outputting the data for sending.

According to the fourth aspect of the present invention, the data processing method can obtain the same effects as those in the first aspect described above.

A non-transitory recording medium storing therein a data processing program according to a fifth aspect of the invention causes a computer to function as components of the data processing device according to any one of the first to third aspects.

According to the fifth aspect of the present invention, the non-transitory recording medium storing therein the data processing program can obtain the same effects as those in the first aspect described above.

According to the present invention, a technology that reduces the amount of biometric data sent can be provided.

### BRIEF DESCRIPTION OF DRAWINGS

FIG. 1 is a block diagram illustrating an application example of a portable terminal according to an embodiment.

FIG. 2 is a block diagram illustrating an example of a data transmission system including a blood pressure monitor and the portable terminal according to an embodiment.

FIG. 3 is a block diagram illustrating an example of the hardware configuration of a blood pressure monitor according to an embodiment.

FIG. 4 is a block diagram illustrating an example of the software configuration of a blood pressure monitor according to an embodiment.

FIG. 5 is a block diagram illustrating an example of the hardware configuration of a portable terminal according to an embodiment. FIG. 6 is a block diagram illustrating an example of the software configuration of a portable terminal according to an embodiment.

FIG. 7 is a flowchart illustrating an example of the output operation of blood pressure data by a portable terminal according to an embodiment.

FIG. 8 is a flowchart illustrating an example of the determination operation of a health-related characteristic by a portable terminal according to an embodiment.

### DESCRIPTION OF EMBODIMENTS

An embodiment according to an aspect of the present invention (hereinafter, also referred to as “the present embodiment”) will be described below with reference to the drawings. However, the present embodiment described below is merely illustrative of the present invention in all respects. Although data mentioned in the description of the present embodiment is described in a natural language, such data may be specifically defined using computer-recognizable languages, e.g., a pseudo language, a command, a parameter, or a machine language. Note that elements that are the same as or similar to the elements described hereinafter are given the same or similar reference signs, and duplicate descriptions thereof will be omitted.

### §1 Application Example

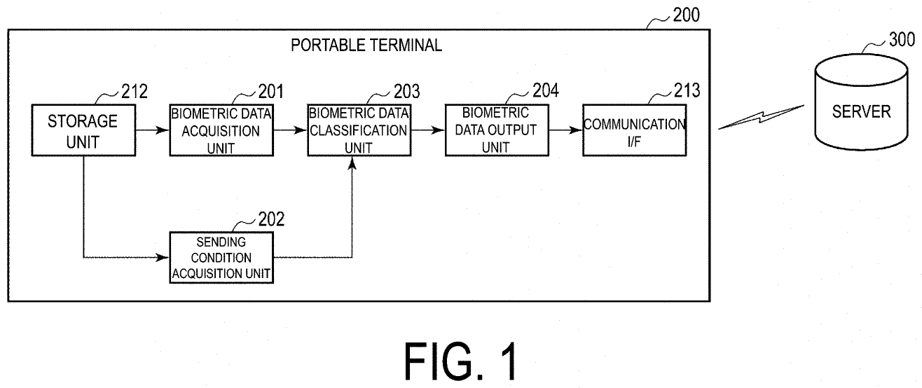

FIG. 1 is a diagram schematically illustrating an application example of a portable terminal 200 according to the present embodiment.

The portable terminal 200 includes a biometric data acquisition unit 201, a sending condition acquisition unit 202, a biometric data classification unit 203, a biometric data output unit 204, a storage unit 212, and a communication interface 213.

The biometric data acquisition unit 201 acquires blood pressure data of a subject from the storage unit 212.

The sending condition acquisition unit 202 references a health-related characteristic of a subject stored in the storage unit 212 and acquires a sending condition that includes a first time period and associated with the health-related characteristic of the subject.

The biometric data classification unit 203 determines whether the measurement time of blood pressure data is within the first time period. The biometric data classification unit 203 classifies blood pressure data as data for sending if the determination result indicates that the measurement time is within the first time period. The biometric data classification unit 203 classifies blood pressure data as data not for sending if the determination result indicates that the measurement time is within a second time period, which is different from the first time period.

The biometric data output unit 204 outputs data for sending to a server 300, i.e., a sending destination, via the communication interface 213.

As described above, according to the present embodiment, the portable terminal 200 can reduce the amount of blood pressure data sent to the server 300.

### §2 Configuration Example

### Data Transmission System

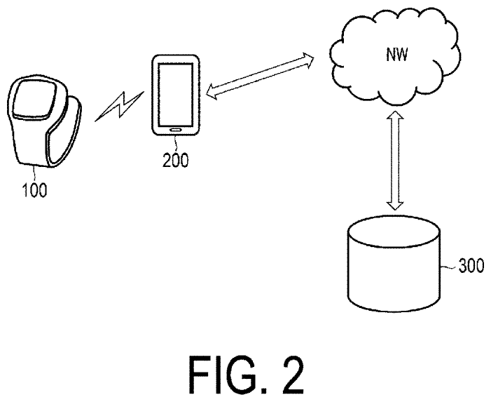

FIG. 2 is a block diagram illustrating an example of a data transmission system including a blood pressure monitor 100 and the portable terminal 200 according to the present embodiment.

The blood pressure monitor 100 is a blood pressure monitor capable of measuring the blood pressure of a subject (user) continuously per heartbeat. For example, the blood pressure monitor 100 is a wearable blood pressure monitor. The blood pressure monitor 100 is an example of a biometric information measuring device. Blood pressure data is an example of biometric data. The blood pressure monitor 100 sends the blood pressure data of a subject to the portable terminal 200 via near-field wireless communication.

The portable terminal 200 is a smart phone or a tablet, for example. The portable terminal 200 is an example of a data processing device. The portable terminal 200 receives the blood pressure data from the blood pressure monitor 100 via near-field wireless communication. The portable terminal 200 sends the blood pressure data to the server 300 via a network.

The server 300 is a storage device that stores the blood pressure data of a large number of subjects. The server 300 receives the blood pressure data from the portable terminal 200 via a network. The server 300 stores the blood pressure data, with the blood pressure data being associated with a subject.

### Blood Pressure Monitor

### Hardware Configuration

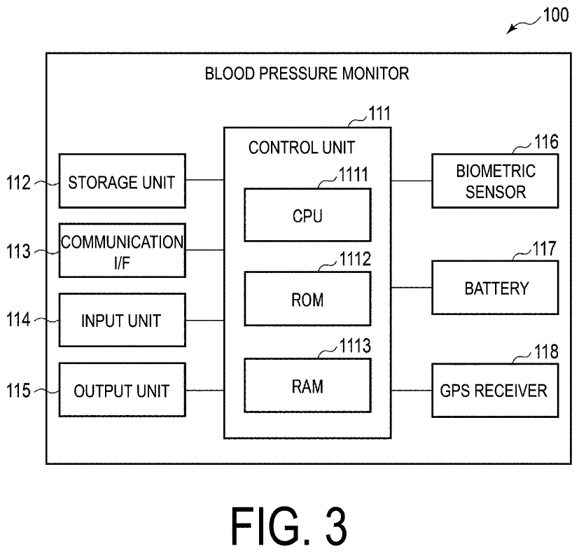

FIG. 3 is a drawing schematically illustrating an example of the hardware configuration of the blood pressure monitor 100.

The blood pressure monitor 100 is a computer that includes a control unit 111, a storage unit 112, a communication interface 113, an input unit 114, an output unit 115, a biometric sensor 116, a battery 117, and a global positioning system (GPS) receiver 118, electrically connected to one another. Note that in FIG. 3, the communication interface is listed as “communication I/F”.

The control unit 111 includes a central processing unit (CPU) 1111, a read-only memory (ROM) 1112, a random access memory (RAM) 1113, and the like. The CPU 1111 is an example of a processor. The CPU 1111 develops a program, which is stored in the storage unit 112, in the RAM 1113. Then, the CPU 1111 interprets and executes the program. This allows the control unit 111 to execute various information processing such as the processing of each block described in the description of the software configuration.

The storage unit 112 is an auxiliary storage device and may be, for example, an internal or external semiconductor memory such as a flash memory, hard disk drive (HDD), or solid state drive (SSD). The storage unit 112 stores programs executed by the control unit 111, data used by the control unit 111, blood pressure data described below, and the like. The program may also be an instruction to operate the control unit 111.

The communication interface 113 includes a module for near-field wireless communication. Near-field wireless communication is, for example, Bluetooth (trade name) communication, but no such limitation is intended. The communication interface 113 communicates directly with the portable terminal 200 using near-field wireless communication.

The input unit 114 is, for example, a device for receiving user input, such as a touch screen, a button, or a switch.

The output unit 115 is a device for outputting, such as a display, a speaker, or the like.

The biometric sensor 116 acquires blood pressure data by measuring the blood pressure of a subject. The operation of the biometric sensor 116 is controlled by a sensor control unit not illustrated, for example. For example, the biometric sensor 116 measures the blood pressure of a subject following a measurement schedule of the blood pressure data stored in the storage unit 112. The measurement schedule defines at least one time period, which is associated with performing blood pressure measurement via the blood pressure monitor 100, in one day. For example, blood pressure data may include values for systolic blood pressure (SBP) and diastolic blood pressure (DBP) and pulse rate, but no such limitation is intended. Furthermore, the blood pressure data includes the time and date of blood pressure measurement. The time and date of measurement is detected by a clock function of the blood pressure monitor 100. Also, the blood pressure data may include the location of blood pressure measurement. The location of blood pressure measurement is detected by the control unit 111 on the basis of the position information of the blood pressure monitor 100 described below.

The biometric sensor 116 may measure the blood pressure of a subject from the pulse transit time (PTT) or via the tonometry method or other technique.

The battery 117 supplies the power supply voltage of the blood pressure monitor 100. The battery 117 may be replaceable.

The GPS receiver 118 receives a plurality of GPS signals send from a plurality of GPS satellites and outputs the GPS signals to the control unit 111. The control unit 111 can calculate the position information of the blood pressure monitor 100 by calculating distance on the basis of the GPS signals. For example, the position information includes information such as latitude and longitude.

Note that, with respect to the specific hardware configuration of the blood pressure monitor 100, it is possible to omit, replace, and add components as appropriate in accordance with embodiments. For example, the control unit 111 may include a plurality of processors. The blood pressure monitor 100 may include a plurality of sensor devices.

### Blood Pressure Monitor

### Software Configuration

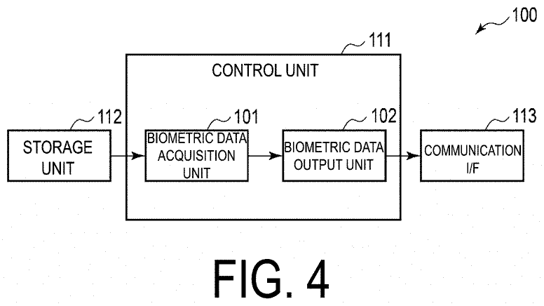

FIG. 4 is a drawing schematically illustrating an example of the software configuration of the blood pressure monitor 100.

The control unit 111 implements a biometric data acquisition unit 101 and a biometric data output unit 102.

The biometric data acquisition unit 101 will be described.

The biometric data acquisition unit 101 acquires blood pressure data from the storage unit 112. The biometric data acquisition unit 101 outputs the blood pressure data to the biometric data output unit 102.

The biometric data output unit 102 will be described.

The biometric data output unit 102 receives blood pressure data from the biometric data acquisition unit 101. The biometric data output unit 102 outputs the blood pressure data to the communication interface 113. In this way, the communication interface 113 sends the blood pressure data to the portable terminal 200 using near-field wireless communication.

### Portable Terminal

### Hardware Configuration

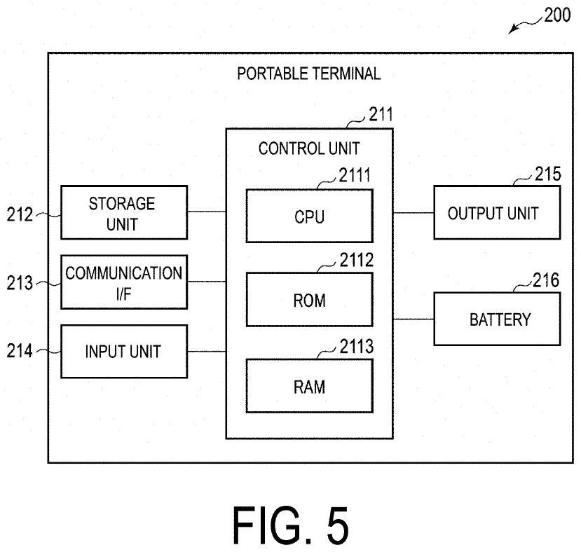

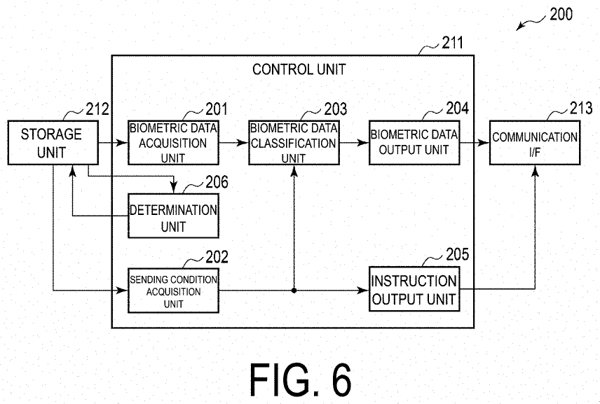

FIG. 5 is a drawing schematically illustrating an example of the hardware configuration of the portable terminal 200.

The portable terminal 200 is a computer that includes a control unit 211, a storage unit 212, a communication interface 213, an input unit 214, an output unit 215, and a battery 216, electrically connected to one another. Note that in FIG. 5, the communication interface is listed as “communication I/F”.

The control unit 211 includes a CPU 2111, a ROM 2112, a RAM 2113, for example. The CPU 2111 is an example of a processor. The CPU 2111 develops a program, which is stored in the storage unit 212, in the RAM 2113. Then, the CPU 2111 interprets and executes the program. This allows the control unit 211 to execute various information processing such as the processing of each block described in the description of the software configuration.

The storage unit 212 is an auxiliary storage device and may be, for example, an internal or external semiconductor memory such as a flash memory. The storage unit 212 stores programs executed by the control unit 211, data used by the control unit 211, and the like. The storage unit 212 stores the blood pressure data the portable terminal 200 receives from the blood pressure monitor 100. The program may also be an instruction to operate the control unit 211.

The communication interface 213 includes various wireless communication modules for near-field wireless communication, mobile communications (3G, 4G, and the like), wireless local area network (WLAN), and the like. Near-field wireless communication is, for example, Bluetooth communication, but no such limitation is intended. The wireless communication module for near-field wireless communication receives blood pressure data from the blood pressure monitor 100. The wireless communication module for mobile communication or the wireless communication for WLAN send the blood pressure data to the server 300 via a network.

The input unit 214 is, for example, a device for receiving user input such as a touch screen.

The output unit 215 is a device for outputting, such as a display, a speaker, or the like.

The battery 216 supplies the power supply voltage of the portable terminal 200. The battery 216 may be replaceable.

Note that, with respect to the specific hardware configuration of the portable terminal 200, it is possible to omit, replace, and add components as appropriate in accordance with embodiments. For example, the control unit 211 may include a plurality of processors.

### Portable Terminal

### Software Configuration

FIG. 6 is a drawing schematically illustrating an example of the software configuration of the portable terminal 200.

The control unit 211 implements the biometric data acquisition unit 201, the sending condition acquisition unit 202, the biometric data classification unit 203, the biometric data output unit 204, an instruction output unit 205, and a determination unit 206.

The biometric data acquisition unit 201 will be described.

The biometric data acquisition unit 201 acquires blood pressure data of a subject from the storage unit 212.

The biometric data acquisition unit 201 outputs the blood pressure data of a subject to the biometric data classification unit 203.

The sending condition acquisition unit 202 will be described.

The sending condition acquisition unit 202 acquires a sending condition that includes a first time period and associated with the health-related characteristic of a subject, as described below. First, the sending condition acquisition unit 202 acquires a health-related characteristic of a subject from the storage unit 212. The health-related characteristic of a subject may be set in advance by the subject, or may be updated as appropriate via a determination by the determination unit 206 described below.

For example, the health-related characteristic of a subject may be symptoms of a specific medical condition. Specific medical conditions include diseases such as cerebral infarction, sleep apnea syndrome, or high blood pressure occurring during a stay at a particular location, but no such limitation is intended. For example, high blood pressure that occurs during a stay at a particular location may be high blood pressure that occurs during stay at work, i.e., work-related high blood pressure.

Next, the sending condition acquisition unit 202 compares the health-related characteristic of the subject against the sending condition database. The sending condition database is a database in which the characteristics relating to health and the sending conditions are associated with one another. The sending condition database is stored in the storage unit 212.

The sending condition is a condition for extracting the blood pressure data, required for analysis by the server 300, from the blood pressure data of a subject. The sending condition includes the time period in which the blood pressure data required for analysis by the server 300 is measured (hereinafter, also referred to as the first time period). The first time period varies depending on the health-related characteristic of a subject. This is because the time period in which blood pressure changes in a distinctive manner varies depending on the health-related characteristic. By the server 300 analyzing the blood pressure data measured in the first time period, the condition of the subject such as the rate of recovery of the health-related characteristic of a subject can be analyzed.

Specific examples of sending conditions will be described. For example, in the case where the health-related characteristic is having symptoms of a cerebral infarction, the sending condition includes a time period in the morning as the first time period. The time period in the morning is, for example, from 5 a.m. to 7 a.m. However no such limitation is intended, and the time period can be set as appropriate. For example, in the case where the health-related characteristic is having symptoms of sleep apnea syndrome, the sending condition includes a time period during sleep as the first time period. The time period during sleep is, for example, from 11 p.m. to 5 a.m. However no such limitation is intended, and the time period can be set as appropriate. For example, in the case where the health-related characteristic is having symptoms of work-related high blood pressure, the sending condition includes a time period during the day as the first time period. The time period during the day is, for example, from 11 a.m. to 3 p.m. However no such limitation is intended, and the time period can be set as appropriate.

Note that the sending condition may include, in addition to the first time period, position information of the location where the blood pressure data required for analysis by the server 300 is measured. For example, in the case where the health-related characteristic is having symptoms of work-related high blood pressure, the sending condition includes position information of a workplace in addition to a time period during the day as the first time period.

The sending condition acquisition unit 202 extracts one or more sending conditions in response to a determination result indicating that one or more sending conditions corresponding to the health-related characteristic of a subject is in the sending condition database. The sending condition acquisition unit 202 outputs the sending condition to the biometric data classification unit 203 and the instruction output unit 205.

The biometric data classification unit 203 will be described.

The biometric data classification unit 203, as described below, determines whether the measurement time of blood pressure data is within the first time period, classifies blood pressure data as data for sending if the determination result indicates that the measurement time is within the first time period, and classifies blood pressure data as data not for sending if the determination result indicates that the measurement time is within the second time period, which is different from the first time period. First, the biometric data classification unit 203 receives blood pressure data from the biometric data acquisition unit 201. The biometric data classification unit 203 receives a sending condition from the sending condition acquisition unit 202. Next, the biometric data classification unit 203 determines whether the measurement time in blood pressure data is within the first time period included in the sending condition. The biometric data classification unit 203 classifies blood pressure data as data for sending if the determination result indicates that the measurement time is within the first time period. Also, the biometric data classification unit 203 classifies blood pressure data as data not for sending if the determination result indicates that the measurement time is within a second time period, which is different from the first time period. For example, the second time period is a time period including all of the time of one day except the time during the first time period.

The biometric data classification unit 203 outputs the data for sending to the biometric data output unit 204. Alternatively, the biometric data classification unit 203 ends processing on the data not for sending. The biometric data classification unit 203 may remove the blood pressure data classified as data not for sending from the storage unit 212.

Note that in a case where the sending condition includes position information in addition to the first time period, the biometric data classification unit 203 executes the processing described below. First, the biometric data classification unit 203 determines whether the measurement time in blood pressure data is within the first time period in the sending condition. The biometric data classification unit 203 classifies blood pressure data as data not for sending if the determination result indicates that the measurement time is within a second time period, which is different from the first time period. The biometric data classification unit 203 compares the measurement location in the blood pressure data against the position information in the sending condition if the determination result indicates that the measurement time is within the first time period. The biometric data classification unit 203 classifies blood pressure data as data for sending if the determination result indicates that measurement location in the blood pressure data matches or roughly matches the position information in the sending condition. The biometric data classification unit 203 classifies blood pressure data as data not for sending if the determination result indicates that measurement location in the blood pressure data does not match nor roughly match the position information in the sending condition.

The biometric data output unit 204 will be described.

The biometric data output unit 204 outputs the data for sending as described below. First, the biometric data output unit 204 receives data for sending from the biometric data classification unit 203. The biometric data output unit 204 outputs data for sending to the server 300, i.e., the sending destination. The biometric data output unit 204 outputs the data for sending to the communication interface 213. In this manner, the communication interface 213 sends the data for sending to the server 300 via a network.

The instruction output unit 205 will be described.

As described below, in the case where at least a portion of the second time period is within the measurement schedule, the instruction output unit 205 outputs an instruction to stop measurement for the blood pressure data in the second time period. First, the instruction output unit 205 receives a sending condition from the sending condition acquisition unit 202. Next, the instruction output unit 205 references the first time period in the sending condition and acquires information relating to the second time period, which is different from the first time period. Next, the instruction output unit 205 acquires the measurement schedule of blood pressure data stored in the storage unit 212. Then, the instruction output unit 205 determines whether at least a portion of the second time period is within the measurement schedule. In the case where at least a portion of the second time period is within the measurement schedule, the instruction output unit 205 generates an instruction to stop measurement for the blood pressure data in the second time period (hereinafter, also referred to as a measurement stop instruction). In one example, the measurement stop instruction includes an instruction to update the measurement schedule so that blood pressure data measurement in the second time period is stopped. In another example, the measurement stop instruction includes a measurement schedule updated so that blood pressure data measurement in the second time period is stopped. The instruction output unit 205 outputs the measurement stop instruction to the blood pressure monitor 100, i.e., the sending destination. The biometric data output unit 204 outputs the measurement stop instruction to the communication interface 213. In this way, the communication interface 213 sends the measurement stop instruction to the blood pressure monitor 100 using near-field wireless communication. On the basis of the measurement stop instruction, the blood pressure monitor 100 updates the measurement schedule stored in the storage unit 112 so that the second time period is removed from at least one time period specified in the measurement schedule. The blood pressure monitor 100 measures the blood pressure of a subject according to the updated measurement schedule. Thus, the blood pressure monitor 100 can stop measurement for blood pressure data in the second time period.

The determination unit 206 will be described.

The determination unit 206, as described below, determines a characteristic relating to the health on the basis of the blood pressure data. First, the determination unit 206 acquires the blood pressure data of a predetermined period from the storage unit 212. For example, the predetermined period is one day. However, no such limitation is intended. Next, the determination unit 206 references the blood pressure data for the predetermined period and extracts the characteristic of the blood pressure of a subject. For example, the characteristic of the blood pressure is a time of blood pressure surge, a time when blood pressure rises to a predetermined value or above, or a period of time over which high blood pressure has continued. However, no such limitation is intended.

Next, the determination unit 206 compares the characteristic of the blood pressure of a subject against the health characteristic database. The health characteristic database is a database in which health-related characteristics and characteristics of blood pressure are associated with one another. The health characteristic database is stored in the storage unit 212.

Specific examples of the health characteristic database will be described. For example, in the case where the health-related characteristic is a symptom of a cerebral infarction, the characteristic of blood pressure is specified as a blood pressure surge occurring a predetermined number of times within a predetermined time period (for example, between 5 a.m. and 7 a.m.). For example, in the case where the health-related characteristic is a symptom of sleep apnea syndrome, the characteristic of blood pressure is specified as a blood pressure being continuously equal to or greater than a predetermined value within a predetermined time period (for example, between 11 p.m. and 5 a.m.) for a predetermined period of time. For example, in the case where the health-related characteristic is a symptom of work-related high bloody pressure, the characteristic of blood pressure is specified as a blood pressure being continuously equal to or greater than a predetermined value within a predetermined time period (for example, between 11 a.m. and 3 p.m.) for a predetermined period of time.

The determination unit 206 determines one or more health-related characteristics in response to a determination result indicating that one or more health-related characteristics corresponding to the characteristic of the blood pressure of a subject is in the health characteristic database. Next, the determination unit 206 stores the determined health-related characteristic in the storage unit 212. In this manner, the storage unit 212 updates and stores the health-related characteristic of a subject.

Note that the time the determination unit 206 determines the health-related characteristic of a subject may be any time and is without limitation.

### §3 Operation Example

### Portable Terminal

### Output Operation of Blood Pressure Data

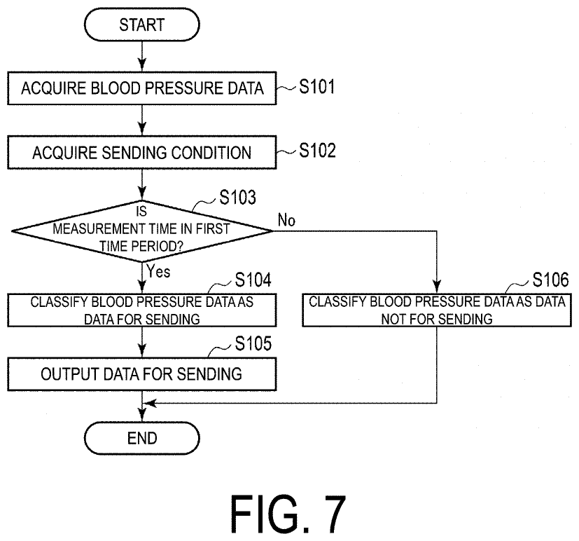

FIG. 7 is a flowchart illustrating an example of the output operation of the blood pressure data by the portable terminal 200. Note that the processing procedure described below is merely an example, and each of the processes may be changed. Furthermore, according to the processing procedure described below, steps may be omitted, replaced, or added as appropriate depending on the embodiment.

As illustrated, the biometric data acquisition unit 201 acquires the blood pressure data of a subject (step S101).

As illustrated, the sending condition acquisition unit 202 acquires a sending condition that includes a first time period and associated with the health-related characteristic of a subject (step S102).

As illustrated, the biometric data classification unit 203 determines whether the measurement time of blood pressure data is within the first time period (step S103). As illustrated, the biometric data classification unit 203 classifies blood pressure data as data for sending (step S104) if the determination result indicates that the measurement time of the blood pressure data is within the first time period (Yes in step S103). As illustrated, the biometric data output unit 204 outputs the data for sending as described below (step S105). In this manner, the portable terminal 200 can send the data for sending to the server 300.

As illustrated, the biometric data classification unit 203 classifies blood pressure data as data not for sending (step S106) if the determination result indicates that the measurement time of the blood pressure data is within a second time period different from the first time period (No in step S103). The biometric data classification unit 203 ends processing of the data not for sending. In this manner, the portable terminal 200 does not send the data not for sending to the server 300.

### Portable Terminal

### Determination Operation of Health-Related Characteristic

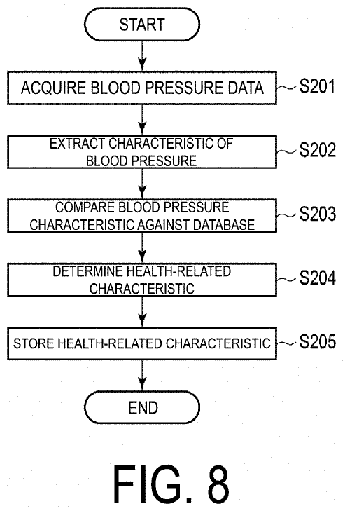

FIG. 8 is a flowchart illustrating an example of the determination operation of the health-related characteristic by the portable terminal 200. Note that the processing procedure described below is merely an example, and each of the processes may be changed. Furthermore, according to the processing procedure described below, steps may be omitted, replaced, or added as appropriate depending on the embodiment.

As illustrated, the determination unit 206 acquires the blood pressure data during a predetermined time period (step S201). As illustrated, the determination unit 206 references the blood pressure data for the predetermined period and extracts the characteristic of the blood pressure of a subject (step S202). As illustrated, the determination unit 206 compares the characteristic of the blood pressure of a subject against the health characteristic database (step S203). As illustrated, the determination unit 206 determines a health-related characteristic of a subject (step S204). As illustrated, the determination unit 206 stores the health-related characteristic of a subject in the storage unit 212 (step S205).

According to this example, the sending condition acquisition unit 202 can acquire a sending condition associated with the health-related characteristic determined by the determination unit 206.

### Actions and Effects

As described above, in the present embodiment, the portable terminal 200 classifies blood pressure data as data for sending if the determination result indicates that the measurement time of the blood pressure data is within the first time period, classifies blood pressure data as data not for sending if the determination result indicates that the measurement time of the blood pressure data is within the second time period, which is different from the first time period, and outputs the data not for sending.

In this manner, the portable terminal 200 can output, to the server 300, blood pressure data of a subject, from among blood pressure data pieces, that satisfies the sending condition and analyzed by the server 300. Thus, the server 300 stores the blood pressure data that satisfies the sending condition and is analyzed but does not store the blood pressure data that does not satisfy the sending condition and is not analyzed. In this manner, the storage capacity of the server 300 can be effectively used.

Furthermore, in the present embodiment, the portable terminal 200 determines the health-related characteristic of a subject on the basis of the blood pressure data.

In this manner, the portable terminal 200 can determine the characteristic related to the current health of a subject on the basis of the blood pressure data that changes over time due to the lifestyle habits of the subject. Thus, the server 300 can store the blood pressure data that satisfies the sending condition associated with the characteristic relating to the current health of a subject.

Furthermore, in the present embodiment, in the case where at least a portion of the second time period is within the measurement schedule, the portable terminal 200 outputs an instruction to stop measurement for the blood pressure data in the second time period.

In this manner, the portable terminal 200 can stop measurement for blood pressure data that does not satisfy the sending condition and not analyzed by the server 300. Thus, the load on measurement processing of the blood pressure data and the load on classification processing of the blood pressure data are reduced.

### §4 Modified Example

### Modified Example 1

In the embodiment described above, the blood pressure monitor continuously monitors blood pressure moment by moment. However, no such limitation is intended. Instead of the blood pressure monitor 100 monitoring continuously moment by moment, the blood pressure monitor 100 may be a blood pressure monitor that performs, in accordance with operation by a subject, a measurement operation at a preset measurement time (hereinafter, also referred to as a non-continuous blood pressure monitor). A non-continuous blood pressure monitor measures the blood pressure of a user using a cuff as a pressure sensor (via the oscillometric method).

### Modified Example 2

In the embodiment described above, blood pressure data is used. However, no such limitation is intended. The embodiment described above, biometric data other than blood pressure data can be used. For example, data that indicates biometric information, such as an electrocardiogram, pulse rate, body temperature, and the like may be used.

### Modified Example 3

In the embodiment described above, the portable terminal 200 is provided. However, no such limitation is intended. For the embodiment described above, a non-portable device that can receive blood pressure data from the blood pressure monitor 100 and send data for sending to the server 300 may be used.

### Modified Example 4

The processing of the portable terminal 200 described above may be performed by the blood pressure monitor 100. In other words, the control unit 111 of the blood pressure monitor 100 may implement the biometric data acquisition unit 201, the sending condition acquisition unit 202, the determination unit 206, the biometric data classification unit 203, the biometric data output unit 204, and the instruction output unit 205. In this example, the portable terminal 200 is an example of a data processing device.

In this example, the storage unit 112 of the blood pressure monitor 100 stores the sending condition database and the health characteristic database. Furthermore, the communication interface 113 of the blood pressure monitor 100 includes the wireless communication module for mobile communication and the wireless communication module for WLAN. In this manner, the blood pressure monitor 100 can send the data for sending to the server 300 via a network.

In this example, the control unit 111 outputs the measurement stop instruction described above to a sensor control unit. On the basis of the measurement stop instruction, the sensor control unit updates the measurement schedule stored in the storage unit 112 so that the second time period is removed from at least one time period specified in the measurement schedule. The sensor control unit measures the blood pressure of a subject according to the updated measurement schedule. Thus, the sensor control unit can stop measurement for blood pressure data in the second time period.

### Other Modified Examples

The present invention is not limited to the embodiments described above, and, in implementation, the components can be modified and embodied within a range that does not depart from the gist of the present invention. Furthermore, various inventions can be formed by appropriately combining a plurality of the components described in the embodiments described above. For example, from among the components described in the embodiments, some components may be omitted. Also, components from different embodiments may be appropriately combined.

### §5 Supplement

Some or all of the embodiments described above can be described as indicated by the following supplement in addition to the scope of the claims. However no such limitation is intended.

### Supplement 1

A data processing device, comprising:

a processor configured to

acquire biometric data of a subject,

acquire a sending condition that comprises a first time period and associated with a health-related characteristic of the subject,

determine whether a measurement time of the biometric data is within the first time period, classify the biometric data as data for sending when a determination result indicates that the measurement time is within the first time period, and classify the biometric data as data not for sending when the determination result indicates that the measurement time is within a second time period, which is different from the first time period, and

output the data for sending; and

a memory in which an instruction to operate the processor is stored.

### Supplement 2

A data processing method, comprising:

using at least one processor to acquire biometric data of a subject;

using the at least one processor to acquire a sending condition that comprises a first time period and associated with a health-related characteristic of the subject;

using the at least one processor, determining whether a measurement time of the biometric data is within the first time period, classifying the biometric data as data for sending when a determination result indicates that the measurement time is within the first time period, and classifying the biometric data as data not for sending when the determination result indicates that the measurement time is within a second time period, which is different from the first time period; and

using the at least one processor to output the data for sending.

### REFERENCE SIGNS LIST

100 . . . Blood pressure monitor

200 . . . Portable terminal

300 . . . Server

101 . . . Biometric data acquisition unit

102 . . . Biometric data output unit

111 . . . Control unit

112 . . . Storage unit

113 . . . Communication interface

114 . . . Input unit

115 . . . Output unit

116 . . . Biometric sensor

117 . . . Battery

118 . . . GPS receiver

201 . . . Biometric data acquisition unit

202 . . . Sending condition acquisition unit

203 . . . Biometric data classification unit

204 . . . Biometric data output unit

205 . . . Instruction output unit

206 . . . Determination unit

211 . . . Control unit

212 . . . Storage unit

213 . . . Communication interface

214 . . . Input unit

215 . . . Output unit

216 . . . Battery

1111 . . . CPU

1112 . . .ROM

1113 . . . RAM

2111 . . . CPU

2112 . . .ROM

2113 . . . RAM

1. A data processing device, comprising:
a processor; and
a memory, wherein
the processor is configured to:
acquire biometric data of a subject;
acquire a sending condition that comprises a first time period and associated with a health-related characteristic of the subject;
determine whether a measurement time of the biometric data is within the first time period, classify the biometric data as data for sending when a determination result indicates that the measurement time is within the first time period, and classify the biometric data as data not for sending when the determination result indicates that the measurement time is within a second time period, which is different from the first time period; and
output the data for sending.

2. The data processing device according to claim 1, wherein
the processor is further configured to determine health-related characteristic on the basis of the biometric data, and
the processor acquires the sending condition associated with the determined health-related characteristic.

3. The data processing device according to claim 1, wherein
the processor is further configured to acquire a measurement schedule of the biometric data and output an instruction to stop measurement for the biometric data in the second time period in a case where at least a portion of the second time period is within the measurement schedule.

4. A data processing method, comprising:
acquiring, with a processor, biometric data of a subject;
acquiring, with the processor, a sending condition that comprises a first time period and associated with a health-related characteristic of the subject;
determining, with the processor, whether a measurement time of the biometric data is within the first time period, classifying, with the processor, the biometric data as data for sending when a determination result indicates that the measurement time is within the first time period, and classifying, with the processor, the biometric data as data not for sending when the determination result indicates that the measurement time is within a second time period, which is different from the first time period; and
outputting, with the processor, the data for sending.

5. A non-transitory recording medium storing therein a data processing program for causing a computer including a processor and a memory to function as the data processing device according to claim 1.

6. The data processing device according to claim 2, wherein
the processor is further configured to acquire a measurement schedule of the biometric data and output an instruction to stop measurement for the biometric data in the second time period in a case where at least a portion of the second time period is within the measurement schedule.

7. A non-transitory recording medium storing therein a data processing program for causing a computer including a processor and a memory to function as components of the data processing device according to claim 2.

8. A non-transitory recording medium storing therein a data processing program for causing a computer including a processor and a memory to function as the data processing device according to claim 3.
